(19) United States
(12) Patent Application Publication
(10) Pub. No.: US 2017/0119738 A1
(43) Pub. Date: May 4, 2017

(54) PHARMACEUTICAL COMPOSITIONS FOR RIZATRIPTAN

(71) Applicant: Dr. Reddy's Laboratories, Ltd., Hyderabad, A.P. (IN)

(72) Inventors: Rajeev Singh RAGHUVANSHI, Gurgaon (IN); Piyush GUPTA, Uttar Pradesh (IN); Rajesh Ramesh PATIL, Maharashtra (IN); Mohan RATHI, Maharashtra (IN)

(21) Appl. No.: 15/337,970

(22) Filed: Oct. 28, 2016

(30) Foreign Application Priority Data
Oct. 28, 2015 (IN) 2670/CHE/2015

(51) Int. Cl. A61K 31/4196; A61K 47/02; A61K 9/00

(52) U.S. Cl. CPC A61K 31/4196; A61K 9/0019; A61K 47/02

(57) ABSTRACT

The present application relates to a method of treating a migraine headache in a patient by administering a pharmaceutical composition including rizatriptan or pharmaceutically acceptable salts thereof contained in a unit dose.

### RELATED APPLICATIONS

This application claims priority from Indian Provisional Application No. 2670/CHE/2015, filed on Oct. 28, 2015, the entire disclosure of which is incorporated herein by this reference.

### TECHNICAL FIELD

The present application relates to a method of treating a migraine headache in a patient by administering a pharmaceutical composition including rizatriptan or pharmaceutically acceptable salts thereof contained in a unit dose. It also provides a method of administering pharmaceutical composition including rizatriptan, which is administered parenterally to a patient suffering from acute migraine with or without aura.

### BACKGROUND

A migraine is a complex neurological condition that involves several changes in the body, including the dilation of blood vessels, inflammation, and activation of pain receptors. Different medicines such as non-steroidal anti-inflammatory drugs (NSAIDs), triptans and ergotamine are used to target each of these mechanisms. Triptans, which are used to treat and not to prevent migraines, work by constricting dilated or widened blood vessels. Most people who have moderate to severe migraines will need a triptan. Currently seven triptans are commercially available in the US, which are, in order of their clinical development: sumatriptan, zolmitriptan, naratriptan, rizatriptan, almotriptan, eletriptan and frovatriptan. All seven, are available in the form of oral tablets. Two triptans (sumatriptan and zolmitriptan) are also available as nasal sprays, one (sumatriptan) is available as a patch, one (sumatriptan) is available as subcutaneous injection and two (rizatriptan and zolmitriptan) are available as orally disintegrating tablets (ODT). An ODT is a drug dosage form, which differs from traditional tablets in that they are designed to be dissolved on the tongue rather than swallowed whole.

Oral administration of triptans is not always the preferred route, especially during migraine-related nausea and migraine-associated gastroparesis, which can further compromise therapeutic efficacy. Similarly, nasal spray preparations are not wholly absorbed in the nasal passages and partly depend on gastrointestinal absorption. They also have a bitter taste, which can be particularly adverse for patients experiencing nausea and/or vomiting and may lead the patient to delay or avoid treatment. Sumatriptan is the only triptan available as a subcutaneous injection. The choice of non-oral drug delivery routes should be customized for the individual patient and the degree of migraine attack.

Route of administration plays an important role in the onset of action for any drugs, including triptans. Injectable administration offers the most rapid and maximum pain relief by triptans, yet it is also associated with a higher incidence of adverse events. The effectiveness of triptans has largely been judged by pain ratings at the one and two hours post drug administration, which has become a standard measurement. Unfortunately, fewer than half of all patients can expect to experience complete freedom from pain within the first two hours after taking a triptan and far fewer studies have examined pain relief over 24 hours. Pain recurrence is also a common complaint of migraine patients. Some people must take second and third doses of their triptan and/or other types of migraine medications to “rescue” themselves from headache pain that returns within 24 hours of the initial relief.

Rizatriptan is a 5-hydroxytryptamine (5-HT₁) agonist, second-generation triptan drug. It has the chemical name N,N-dimethyl-2-[5-(1H-1,2,4-triazol-1-ylmethyl)-1H-indol-3-yl]ethanamine, the empirical formula C₁₅H₁₉N₅, and a molecular weight of 269.4. It was developed by Merck & Co., for the treatment of acute migraine attacks with or without aura. Rizatriptan is available in strengths of 5 and 10 mg as conventional tablets (MAXALT®) and orally disintegrating tablets (MAXALT-MLT®) in the United States. Rizatriptan can refer to rizatriptan benzoate, and is often used to refer to the rizatriptan salt, but can also be used to refer to the rizatriptan base active.

Rizatriptan binds with high affinity to human cloned 5-HT_{1B/1D} receptors. MAXALT® and MAXALT-MLT® presumably exert their therapeutic effects in the treatment of migraine headaches by binding to 5-HT_{1B/1D} receptors located on intracranial blood vessels and sensory nerves of the trigeminal system.

Various comparative studies for triptan effectiveness, as mentioned below, show rizatriptan as better choice among other available triptans.

D. S. Ng-Mak et al. (Int. J Clin. Pract., Volume 61, Issue 7, pages 1091-1111, July 2007) have reported that rizatriptan (10 mg) is significantly more effective than other oral triptans for pain relief (PR) and pain freedom (PF). CONSUMER REPORTS® Best Buy Drugs (March 2013) reports that, as compared to treatment with the 50 mg tablet of sumatriptan, the orally dissolvable tablet form of rizatriptan (10 mg) provides complete freedom from pain and return to normal function at two hours, and there were more people with sustained freedom from pain at 24 hours.

J. U. Adelman et al. (Neurology, Volume 57, pages 1377-1383, 2001) compared the efficacy of oral rizatriptan (10 mg) with oral doses of sumatriptan, naratriptan, and zolmitriptan and found that oral rizatriptan (10 mg) was more effective on stringent outcome measures of pain-free response at 2 hours, symptom-free response at 2 hours, and 24 hour sustained pain-free response.

However, commercially available orally disintegrating rizatriptan dosage form has been shown to trigger migraine headache due to the presence of aspartame. Lawrence C. et al. (Headache: The Journal of Head and Face Pain, Volume 41, Issue 9, pages 899-901, October 2001) reported that rizatriptan ODT contains the artificial sweetener aspartame, which may trigger a migraine in those sensitive to it.

Additionally, according to CONSUMER REPORTS® Best Buy Drugs (March 2013), doctors advise against mixing of triptans. Thus, if a patient is on another triptan but needs faster, reliable relief for an unusually severe or persistent migraine, he/she must not switch over to another triptan. Considering this, rizatriptan-responsive patients have very limited options for rizatriptan, which is only available as a conventional oral tablet and an ODT; distinctly, sumatriptan is provided in injectable, nasal spray, patch, and tablet formulations.

In view of the aforementioned references, due to non-availability of rizatriptan dosage forms other than oral route, there is a need to provide rizatriptan in a form that provides for effective, more rapid delivery, such as by parenteral administration.

There is also a need for non-oral, fast acting rizatriptan dosage forms in order to overcome the above mentioned drawbacks related to commercially available rizatriptan dosage forms and to improve patient compliance for the treatment of cluster headaches or migraine headaches with more effective and rapid pain relief.

Several attempts in the past achieved fast pain relief using various delivery systems for triptans. U.S. Patent Application No. 2011/118189 A1 from Zogenix Inc. relates to a combination of a long acting triptan (naratriptan or frovatriptan) and a short acting triptan (sumatriptan, almotriptan, eletriptan, rizatriptan, or zomitriptan), which can be delivered using needle-free delivery system.

International Patent Application No. WO 2012/075209 A1 from Lanco Biosciences Inc. relates to a system comprising a microinjection device and a triptan or triptan-sulfa (sumatriptan) formulation for delivering to subjects via subcutaneous, transdermal, or intradermal delivery. The formulation can be delivered to the subject in a length of time between about 0.1 seconds and 10 minutes.

Nevertheless, there seem to be no parenteral dosage form exists for rizatriptan to ameliorate presently available migraine treatments. Accordingly, the present application provides pharmaceutical compositions including rizatriptan, which are appropriate for parenteral administration. The present application further provides methods for treating cluster headache or migraine headache in a patient, which involve administration of the pharmaceutical compositions. The present application further provides suitable parenteral delivery systems, such as injector devices, for delivery of the pharmaceutical compositions. Accordingly the subject matter of the present application accomplishes unmet needs in the area of such treatments.

### SUMMARY OF THE APPLICATION

In an embodiment, the present application relates to a method of treating a migraine headache in a patient.

In another embodiment, the present application relates to a method of treating a migraine headache in a patient by administering a pharmaceutical composition including rizatriptan or pharmaceutically acceptable salts thereof contained in a unit dose.

In an aspect of the above embodiment, said unit dose comprises at least about 60% less rizatriptan as compared to commercially available oral rizatriptan compositions.

In another embodiment, the present application relates to a method of treating a migraine headache in a patient by administering a pharmaceutical composition including rizatriptan or pharmaceutically acceptable salts thereof, wherein said composition upon administration exhibits a Tₘₐₓ of not more than about 12 minutes.

In an aspect of the above embodiments, the present pharmaceutical composition exhibits a Tₘₐₓ of about 8 minutes to about 12 minutes.

In an aspect of the above embodiment, said unit dose comprises at least about 80% less rizatriptan as compared to commercially available oral rizatriptan compositions.

In another aspect of the above embodiments, said unit dose comprises less than about 5 mg of rizatriptan base.

In yet another aspect of the above embodiments, said unit dose comprises less than about 4 mg of rizatriptan base.

In another embodiment, the present application relates to a method of treating a migraine headache in a patient by administering a pharmaceutical composition including rizatriptan contained in a unit dose, wherein said composition provides at least about 70% of headache relief in about 30 minutes.

In an aspect of the above embodiments, the composition of the present application provides at least about 80% of headache relief in about 60 minutes.

In another aspect of the above embodiments, the present application relates to a method of treating a migraine headache in a patient comprising administering a pharmaceutical composition including rizatriptan contained in a unit dose, wherein said unit dose is a single use parenteral injector device.

In another aspect of the above embodiments, said composition is parenterally administered.

In another aspect of the above embodiments, said unit dose is provided in a single use parenteral auto-injector device designed for subcutaneous or intramuscular injection.

In yet another aspect of the above embodiments, said composition further comprises at least one stabilizing agent comprising sodium chloride.

In an embodiment, the present application relates to a method of administering a pharmaceutical composition including rizatriptan or pharmaceutically acceptable salts thereof contained in a unit dose to a patient in need thereof, wherein said composition upon administration exhibits at least one of the following pharmacokinetic parameters:

a. Tmax value of not more than about 12 minutes;
b. Cₘₐₓ of not more than about 37 ng/ml;
c. AUC₀₋₂ₕ of not more than about 31 ng·hr/ml;
d. AUC₀₋ₜ of not more than about 44 ng·hr/ml; and
e. AUC_{0-refTmax} of not more than about 19 ng·hr/ml.

In an aspect of the above embodiments, said unit dose comprises less than about 5 mg of rizatriptan base.

In another aspect of the above embodiments, the composition of the present application exhibits AUC_{0-refTmax} of at least about 90% higher than commercially available oral rizatriptan compositions.

In another aspect of the above embodiments, the composition of the present application exhibits AUC_{0-refTmax} of at least about 70% higher as compared to commercially available oral rizatriptan composition.

In another aspect of the above embodiments, the composition of the present application exhibits AUC_{0-refTmax} of at least about 60% higher as compared to commercially available oral rizatriptan composition.

In another aspect of the above embodiments, the composition of the present application is contained in a unit dose, wherein said composition upon administration to patients exhibits a different response between genders, wherein the female patients are more responsive than the male patients.

In another aspect of the above embodiments, the composition of the present application exhibits at least about a 90% lower Tₘₐₓ value for female patients as compared to male patients.

In another aspect of the above embodiments, the composition of the present application exhibits at least about an 80% lower Tₘₐₓ value for female patients as compared to male patients.

In another aspect of the above embodiments, the composition of the present application exhibits at least about a 60% lower Tₘₐₓ value for female patients as compared to male patients.

In another aspect of the above embodiments, the composition of the present application exhibits at least about a 90% higher Cₘₐₓ value for female patients as compared to male patients.

In another aspect of the above embodiments, the composition of the present application exhibits at least about an 80% higher Cₘₐₓ value for female patients as compared to male patients.

In another aspect of the above embodiments, the composition of the present application exhibits at least about a 70% higher Cₘₐₓ value for female patients as compared to male patients.

In another aspect of the above embodiments, the composition of the present application exhibits at least about a 60% higher Cₘₐₓ value for female patients as compared to male patients.

In yet another aspect of the above embodiments, the composition of the present application exhibits at least about 90% higher AUC₀₋₂ₕ, AUC₀₋ₜ or AUC_{0-refTmax} values for female patients as compared to male patients.

In yet another aspect of the above embodiments, the composition of the present application exhibits at least about 80% higher AUC₀₋₂ₕ, AUC₀₋ₜ or AUC_{0-refTmax} values for female patients as compared to male patients.

In yet another aspect of the above embodiments, the composition of present application exhibits at least about 70% higher AUC₀₋₂ₕ, AUC₀₋ₜ or AUC_{0-refTmax} values for female patients compared to male patients.

In yet another aspect of the above embodiments, the composition of present application exhibits at least 50% higher AUC₀₋₂ₕ, AUC₀₋ₜ or AUC_{0-refTmax} values for female patients compared to male patients.

In an aspect of the above embodiments, the present application relates to a method of treating a migraine headache in a patient comprising administering a pharmaceutical composition including rizatriptan contained in a unit dose, wherein said unit dose is provided in a single use parenteral injector device.

In another aspect of the above embodiments, the composition is parenterally administered.

In another aspect of the above embodiments, said unit dose is provided in a single use parenteral auto-injector device designed for subcutaneous or intramuscular injection.

In yet another aspect of the above embodiments, the present pharmaceutical composition further comprises at least one stabilizing agent comprising sodium chloride.

In an embodiment, the present application relates to a pharmaceutical composition comprising an aqueous solution of rizatriptan or pharmaceutically acceptable salts thereof and at least one stabilizing agent comprising sodium chloride contained in a unit dose.

In an embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0.

In an aspect of the above embodiments, the present pharmaceutical composition including rizatriptan is contained in a unit dose comprising less than about 5 mg of rizatriptan base.

In another embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0, and said composition has an osmolality of from about 250 mOsmol/kg to about 350 mOsmol/kg.

In another embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0, and said composition has a pH value of about 4.0 to about 7.5.

In an aspect of the above embodiments, the composition comprising an aqueous solution of rizatriptan is stable for at least 6 months upon storage at 25° C. and 60% relative humidity (RH) or 40° C. and 75% relative humidity (RH).

In an aspect of the above embodiments, the composition comprising an aqueous solution of rizatriptan is filterable through 0.2μ membrane filter.

In an aspect of the above embodiments, the composition comprising an aqueous solution of rizatriptan is contained in a unit dose, wherein said unit dose is provided in a single use parenteral injector device.

In another aspect of the above embodiments, said unit dose is provided in a single use parenteral auto-injector device designed for subcutaneous or intramuscular injection.

In another aspect of the above embodiments, the present composition is prepared for administration to a patient suffering from acute migraine with or without aura.

In an embodiment, the present application relates to an auto-injector device comprising a unit dose of the pharmaceutical composition, as disclosed herein.

### BRIEF DESCRIPTION OF THE DRAWING

FIG. 1 shows 8 hour plasma rizatriptan concentration vs. time profile for an exemplary composition of the present application, as set forth in Example 9, vis-à-vis 10 mg of MAXALT® administered to 12 healthy human subjects in fasting conditions.

### DETAILED DESCRIPTION OF THE EMBODIMENTS

The details of one or more embodiments of the present invention are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Definitions: The terms as used herein have the following meanings:

The present invention can comprise or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. All ranges recited herein include the endpoints, including those that recite a range “between” two values.

The terms “a” and “the” as used herein are understood to encompass the plural as well as the singular, or otherwise as clearly mentioned wherever needed. For example, reference to “an excipient” includes reference to one or more of such excipients, and reference to “the vehicle” includes reference to one or more of such vehicles.

The terms such as “about”, “up to”, “generally” and the like are to be construed as modifying a term or value such that it is not an absolute. Such terms will be defined by the circumstances and the terms that they modify as those terms are understood by those of skill in the art. This includes, at very least, the degree of expected experimental error, technical error, and instrumental error for a given experiment, technique or an instrument used to measure a value. The term “about” is used to provide flexibility to a numerical range endpoint by providing that a given value may be “a little above” or “a little below” the endpoint. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum.

The terms “composition” and “formulation” are used interchangeably and refer to a mixture of two or more compounds, elements, or molecules. Also these terms may be used to refer to a mixture of one or more active agents with a pharmaceutically acceptable vehicle or excipients. Furthermore, the term “dosage form” can include one or more formulation(s) or composition(s) provided in a format for administration to a subject.

The terms “drug” and “pharmaceutical” are used interchangeably to refer to a pharmacologically active substance or composition. The terms “parenteral injection” and “parenteral administration” are also used interchangeably. These terms of art are well-known in the pharmaceutical and medicinal arts. Parenteral administration is to be distinguished from enteral administration. Enteral administration involves the gastrointestinal tract and includes, for example, oral administration. Distinctly, parenteral administration excludes enteral routes and includes, for example, subcutaneous, intramuscular, and intravenous administration.

The term “pharmaceutically acceptable salts” as used herein, includes those salts which are, within the scope of sound medical judgment, suitable for use in contact with the tissues of humans and lower animals without undue toxicity, irritation, allergic response and the like, which are well known in the art. The salts can be prepared in situ during the final isolation and purification of the compounds of the invention, or can be prepared separately by reacting the pharmaceutically active substance having a free base function with a suitable organic acid or inorganic acid.

The term “unit dose” as used herein, means a discrete amount of a composition comprising a therapeutically effective amount of rizatriptan or pharmaceutically acceptable salts thereof, sufficient to reduce the migraine, but low enough to avoid serious side effects (at a reasonable benefit/risk ratio), within the scope of sound medical judgment. The effective amount of the rizatriptan or pharmaceutically acceptable salts thereof will vary with the particular condition being treated, the age and physical condition of the patient being treated, the severity of the condition, the duration of the treatment, the nature of concurrent therapy, and like factors within the knowledge and expertise of the attending physician.

As used herein the term “injector device” refers to a medical device designed to deliver a unit dose as disclosed herein. In this regard, in some embodiments the injector device is designed to deliver, as well as to contain and store, the unit dose. Injector devices can be spring-loaded syringes, in some embodiments, and are designed for ease if use, such that self-administration (parenteral) by a patient or parenteral administration by a medically-untrained individual can be readily accomplished. Examples of injector devices include, but are not limited to, auto-injector, syringe like injector, needle free injector, and jet injector devices.

As used herein the term “patient” refers to a target of administration. The term does not denote a particular age or sex. Thus, the term is inclusive of adults and children, whether male or female.

The term “migraine” as used herein is a condition characterized by recurrent attacks of headache, with or without aura (visual or sensory symptoms), that vary widely in intensity, frequency and duration. The term “migraine” also includes, acute migraine with or without heache, chronic migraine, episodic migraine, ophthalmoplegic migraine, basilar migraine, hemiplegic migraine, or generally denoted migraines by the physicians, like but not limited to, stress migraine, silent migraine, sinus migraine, ocular migraine, sesonal migraine, cyclic migraine, gastric stasis migraine, tension migraine, menstrual migraine, and the like. As per International Headache Society (IHS) guidelines a verbal 4-point scale questionnaire is used to measure migraine pain intensity in controlled clinical trials of drugs in migraine conditions: 0—no pain; 1—mild pain (does not interfere with usual activities); 2—moderate pain (inhibits but does not wholly prevent usual activities); 3—severe pain (prevents all activities).

The term “pain relief or headache relief” as used herein, defined as a reduction of headache severity from grades 2 or 3 (moderate or severe) at the time of administration of the dosage form to grades 0 or 1 (no pain or mild pain) at the time point in question.

The term “Tₘₐₓ” as used herein refers to the time point when the maximum concentration (or “Cₘₐₓ”) of the dosage form is observed, post administration.

The term “area under curve (AUC)” as used herein refers to the area under the plasma drug concentration-time curve, which reflects the actual body exposure to drug after administration of a dose of the drug and is expressed in h*ng/ml.

The term “stable” as used herein, refers to a chemical and physical stability of the present pharmaceutical composition including rizatriptan, which remains as clear and colorless liquid, wherein the drug is present in an amount of at least about 95% to about 100% of the originally specified amount and total impurity of not more than about 1.5% for at least about 6 months upon storage at 25° C./60% relative humidity (RH) or at 40° C./75% relative humidity (RH).

The term “commercially available oral rizatriptan composition(s)” as used herein, refers to MAXALT® oral tablets containing rizatriptan benzoate or its pharmaceutical equivalents or its therapeutic equivalents or later approved drugs which are designated as AB rated by US FDA as per Approved Drug Products with Therapeutic Equivalence Evaluations (34th edition) or drugs having obtained marketing approval by US FDA through Abbreviated New Drug Application (ANDA) filing by establishing bioequivalence to such Product. For example, in some embodiments, MAXALT® includes compressed tablet of rizatriptan benzoate along with excipients such as lactose monohydrate, microcrystalline cellulose, pregelatinized starch, ferric oxide (red), and magnesium stearate. In some embodiments MAXALT® includes its US FDA approved therapeutic or pharmaceutical equivalents. MAXALT® is a Trademark registered by Merck & Co., Inc. Corporation New Jersey One Merck Drive Whitehouse Station N.J. 088890100 and owned by Merck Sharp & Dohme Corp. Corporation New Jersey One Merck Drive Whitehouse Station N.J. 088890100.

As used herein, the terms “optional” or “optionally” mean that the subsequently described event or circumstance does or does not occur or exist and that the description includes instances where said event or circumstance occurs or exists and instances where it does not.

As used herein, the terms “treatment” or “treating” relate to curing or substantially curing a condition, as well as ameliorating at least one symptom of the condition, and are inclusive of prophylactic treatment and therapeutic treatment. As would be recognized by one of ordinary skill in the art, if the treatment is administered prior to clinical manifestation of a condition then the treatment is prophylactic (i.e., it protects the subject against developing the condition). If the treatment is administered after manifestation of the condition, the treatment is therapeutic (i.e., it is intended to diminish, ameliorate, control, or maintain the existing condition and/or side effects associated with the condition). The terms relate to medical management of a subject with the intent to substantially cure, ameliorate, stabilize, or substantially prevent a condition, including but not limited to prophylactic treatment to preclude, avert, obviate, forestall, stop, or hinder something from happening, or reduce the severity of something happening, especially by advance action. Thus, prophylactic treatment does not require a complete and absolute prevention of all symptoms associated with the condition. As such, the terms treatment or treating include, but are not limited to: inhibiting the progression of a condition of interest; arresting or preventing the development of a condition of interest; reducing the severity of a condition of interest; ameliorating or relieving symptoms associated with a condition of interest; causing a regression of the condition of interest or one or more of the symptoms associated with the condition of interest; and preventing a condition of interest or the development of a condition of interest.

In an embodiment, the present application relates to a method of treating a migraine headache in a patient.

In another embodiment, the present application relates to a method of treating a migraine headache in a patient by administering a pharmaceutical composition including rizatriptan or pharmaceutically acceptable salts thereof contained in a unit dose.

In an embodiment, the present application relates to a method of treating a migraine headache in a patient by administering a pharmaceutical composition including rizatriptan or pharmaceutically acceptable salts thereof contained in a unit dose, wherein said unit dose comprises at least about 60% less rizatriptan as compared to commercially available oral rizatriptan compositions.

In another embodiment, the present application relates to a method of treating a migraine headache in a patient by administering a pharmaceutical composition including rizatriptan or pharmaceutically acceptable salts thereof contained in a unit dose, wherein said composition upon administration exhibits a Tₘₐₓ of not more than about 12 minutes, and said unit dose comprises at least about 50% less rizatriptan as compared to commercially available oral rizatriptan compositions.

In another embodiment, the present application relates to a method of treating a migraine headache in a patient by administering a pharmaceutical composition including rizatriptan or pharmaceutically acceptable salts thereof contained in a unit dose, wherein said composition upon administration exhibits a Tₘₐₓ of not more than about 12 minutes, and said unit dose comprises at least about 60% less rizatriptan as compared to commercially available oral rizatriptan compositions.

In an aspect of the above embodiments, the present pharmaceutical composition including rizatriptan contained in a unit dose comprises less than about 5 mg of rizatriptan base.

In another aspect of the above embodiments, the present pharmaceutical composition including rizatriptan contained in a unit dose comprises less than about 4 mg of rizatriptan base.

In another aspect of the above embodiments, the present pharmaceutical composition including rizatriptan contained in a unit dose comprises about 3 mg of rizatriptan base.

In an embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base, wherein said composition upon administration exhibits a Tₘₐₓ of not more than about 12 minutes, and said unit dose comprises at least about 50% less rizatriptan as compared to commercially available oral rizatriptan compositions.

In another embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base, wherein said composition upon administration exhibits a Tₘₐₓ of not more than about 12 minutes, and said unit dose comprises at least about 60% less rizatriptan as compared to commercially available oral rizatriptan compositions.

In an aspect of the above embodiments, the composition of the present application exhibits a Tₘₐₓ value of at least about 80% less as compared to commercially available oral rizatriptan compositions.

In another aspect of the above embodiments, the composition of the present application exhibits a Tₘₐₓ value of at least about 80%, 75%, 70%, 65%, or 60% less as compared to commercially available oral rizatriptan compositions.

In an aspect of the above embodiments, the present pharmaceutical composition exhibits a Tₘₐₓ value of about 8 minutes to about 12 minutes.

The pharmacodynamic parameter “% headache relief of the present pharmaceutical composition” is calculated through a validated exposure-response simulation model. Tokuoka et al. (The Journal of Headache and Pain 2014, 15:85) describes an exposure-response model, which shows sigmoidal-Eₘₐₓ relationship between composite index of drug binding rate (Φₘₐₓ/Tₘₐₓ) and binding exposure (AUC_{Φ}) i.e. Φₘₐₓ/Tₘₐₓ*AUC_{Φ} and % headache relief as mentioned below,

E=Eₘₐₓ·Cγ/(EC₅₀γ+Cγ),

where C=Φₘₐₓ/Tₘₐₓ·AUC_{Φ},

E=headache relief rate (%),

Eₘₐₓ=maximum headache relief rate (%),

EC₅₀=value of C when Eₘₐₓ is 50%, and

γ=Hill coefficicent

The pharmacodynamic parameter “% headache relief for MAXALT® 10 mg oral tablet” is simulated from Tokuoka's exposure-response model and the simulated values are validated against % headache relief values available from US FDA approved label for MAXALT® 10 mg oral tablet.

In an embodiment, the present application relates to a method of treating a migraine headache in a patient by administering a pharmaceutical composition including rizatriptan contained in a unit dose, wherein said composition provides headache relief within about 30 minutes comparable to about 120 minutes with MAXALT® 10 mg oral tablets, when simulated from a validated exposure-response model.

In an embodiment, the present application relates to a method of treating a migraine headache in a patient by administering a pharmaceutical composition including rizatriptan contained in a unit dose, wherein said composition provides a pharmacodynamic parameter, such as % headache relief, within about 60 minutes comparable to about 240 minutes with MAXALT® 10 mg oral tablets, when simulated from a validated exposure-response model.

In an aspect of the above embodiments, the present application relates to a method of treating migraine headache in a patient by administering a pharmaceutical composition of rizatriptan contained in a unit dose, wherein said composition provides at least about 70% of headache relief in about 30 minutes.

In an aspect of the above embodiments, the present application relates to a method of treating a migraine headache in a patient by administering a pharmaceutical composition including rizatriptan contained in a unit dose, wherein said composition provides at least about 70% of headache relief in about 30 minutes.

In another embodiment, the present application relates to a method of treating migraine headache in a patient by administering a pharmaceutical composition of rizatriptan or pharmaceutically acceptable salts thereof contained in a unit dose comprising less than about 5 mg of rizatriptan base, wherein said composition provides at least about 80% of headache relief in about 60 minutes.

In another aspect of the above embodiments, the present application relates to a method of treating a migraine headache in a patient by administering a pharmaceutical composition including rizatriptan contained in a unit dose, wherein said composition provides at least about 80% of headache relief in about 60 minutes.

In another embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base, wherein said composition provides at least about 70% of headache relief in about 30 minutes.

In another embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base, wherein said composition provides at least about 80% of headache relief in about 60 minutes.

In yet another aspect of the above embodiments, the present application relates a method of treating a migraine headache in a patient by administering a pharmaceutical composition including rizatriptan contained in a unit dose, wherein said composition provides about four fold faster headache relief as compared to commercially available oral rizatriptan compositions.

In another aspect of the above embodiments, the present application relates a method of treating a migraine headache in a patient by administering a pharmaceutical composition including rizatriptan contained in a unit dose, wherein said composition provides at least about three fold faster headache relief as compared to commercially available oral rizatriptan compositions.

In another aspect of the above embodiments, the present application relates a method of treating a migraine headache in a patient by administering a pharmaceutical composition including rizatriptan contained in a unit dose, wherein said composition provides at least about two fold faster headache relief as compared to commercially available oral rizatriptan compositions.

In an embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base, wherein said composition upon administration exhibits a Tₘₐₓ value of not more than about 12 minutes and provides at least about 70% of headache relief in about 30 minutes.

In another embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base, wherein said composition upon administration exhibits a Tₘₐₓ value of not more than about 12 minutes and provides at least about 80% of headache relief in about 60 minutes.

In an aspect of the above embodiments, said unit dose comprises at least about 60% less rizatriptan as compared to commercially available oral rizatriptan compositions.

In an aspect of the above embodiments, said unit dose is provided in a single use parenteral injector device.

In an aspect of the above embodiments, the present unit dose is parenterally administered using injectable device selected from auto-injector, syringe like injector, needle free injector or j et injector.

In another aspect of the above embodiments, the unit dose is provided in a single use parenteral auto-injector device designed for subcutaneous or intramuscular injection.

In another aspect of the above embodiments, said composition is prepared for administering to a patient suffering from acute migraine with or without aura.

In another aspect of the above embodiments, the present pharmaceutical composition further comprises at least one stabilizing agent.

In yet another aspect of the above embodiments, the present pharmaceutical composition further comprises at least one stabilizing agent comprising sodium chloride.

In an embodiment, the present application relates to a method of treating a migraine headache in a patient by administering a pharmaceutical composition including rizatriptan or pharmaceutically acceptable salts thereof contained in a unit dose comprising less than about 5 mg of rizatriptan base and at least one stabilizing agent comprising sodium chloride, wherein said composition upon administration exhibits a Tₘₐₓ value of not more than about 12 minutes.

In an embodiment, the present application relates to a method of administering a pharmaceutical composition including rizatriptan or pharmaceutically acceptable salts thereof contained in a unit dose to a patient in need thereof, wherein said unit dose comprises less than about 5 mg of rizatriptan base and said composition upon administration exhibits at least one of the following pharmacokinetic parameters:

a. Tₘₐₓ value of not more than about 12 minutes;

b. Cₘₐₓ of not more than about 37 ng/ml;

c. AUC₀₋₂ₕ of not more than about 31 ng·hr/ml;

d. AUC₀₋ₜ of not more than about 44 ng·hr/ml; and

e. AUC_{0-refTmax} of not more than about 19 ng·hr/ml.

In an embodiment, the present application relates to a method of administering a pharmaceutical composition including rizatriptan or pharmaceutically acceptable salts thereof contained in a unit dose to a patient in need thereof, wherein said unit dose comprises less than about 5 mg of rizatriptan base and said composition upon administration exhibits at least one of the following pharmacokinetic parameters:

a. Tₘₐₓ value of not more than about 12 minutes;

b. Cₘₐₓ of about 27 ng/ml to about 37 ng/ml;

c. AUC₀₋₂ₕ of about 23 ng·hr/ml to about 31 ng·hr/ml;

d. AUC₀₋ₜ of about 32 ng·hr/ml to about 44 ng·hr/ml; and

e. AUC_{0-refTmax} of about 13 ng·hr/ml to about 19 ng·hr/ml.

In an aspect of the above embodiments, the present pharmaceutical composition including rizatriptan contained in a unit dose comprises less than about 5 mg of rizatriptan base.

In an aspect of the above embodiments, the present pharmaceutical composition including rizatriptan contained in a unit dose comprises less than about 4 mg of rizatriptan base.

In an aspect of the above embodiments, the present pharmaceutical composition including rizatriptan contained in a unit dose comprises about 3 mg of rizatriptan base.

In an aspect of the above embodiments, the present pharmaceutical composition including rizatriptan contained in a unit dose, exhibits AUC_{0-refTmax} of at least about 90% higher than commercially available oral rizatriptan compositions.

In another aspect of the above embodiments, the composition of present application exhibits AUC_{0-refTmax} of at least about 70% higher than commercially available oral rizatriptan composition.

In another aspect of the above embodiments, the composition of present application exhibits AUC_{0-refTmax} of at least about 60% higher than commercially available oral rizatriptan composition.

In an aspect of the above embodiments, the present pharmaceutical composition including rizatriptan contained in a unit dose, wherein said composition upon administration to the patients exhibits pharmacokinetic variations in male and female patients.

In another aspect of the above embodiments, the present pharmaceutical composition including rizatriptan contained in a unit dose, wherein said composition upon administration to the female patients exhibits pharmacokinetic variations compared to male patients.

In another aspect of the above embodiments, the present pharmaceutical composition including rizatriptan contained in a unit dose upon administration to the female patients exhibits at least about a 90% lower Tₘₐₓ value compared to male patients.

In another aspect of the above embodiments, the present pharmaceutical composition of rizatriptan contained in a unit dose upon administration to the female patients exhibits at least about a 80% lower Tₘₐₓ value compared to male patients.

In another aspect of the above embodiments, the present pharmaceutical composition of rizatriptan contained in a unit dose upon administration to the female patients exhibits at least about a 70% lower Tₘₐₓ value compared to male patients.

In another aspect of the above embodiments, the present pharmaceutical composition of rizatriptan contained in a unit dose upon administration to the female patients exhibits at least about 60% less Tmax value compared to male patients.

In another aspect of the above embodiments, the present pharmaceutical composition including rizatriptan contained in a unit dose upon administration to the female patients exhibits at least about a 90% higher Cₘₐₓ value compared to male patients.

In another aspect of the above embodiments, the present pharmaceutical composition of rizatriptan contained in a unit dose upon administration to the female patients exhibits at least about a 80% higher Cₘₐₓ value compared to male patients.

In another aspect of the above embodiments, the present pharmaceutical composition of rizatriptan contained in a unit dose upon administration to the female patients exhibits at least about a 70% higher Cₘₐₓ value compared to male patients.

In another aspect of the above embodiments, the present pharmaceutical composition of rizatriptan contained in a unit dose upon administration to the female patients exhibits at least about a 60% higher Cₘₐₓ value compared to male patients.

In yet another aspect of the above embodiments, the present pharmaceutical composition including rizatriptan contained in a unit dose upon administration to the female patients exhibits at least about 90% higher AUC₀₋₂ₕ, AUC₀₋ₜ or AUC_{0-refTmax} values as compared to male patients.

In yet another aspect of the above embodiments, the present pharmaceutical composition of rizatriptan contained in a unit dose upon administration to the female patients exhibits at least about 80% higher AUC₀₋₂ₕ, AUC₀₋ₜ or AUC_{0-refTmax} values compared to male patients.

In yet another aspect of the above embodiments, the present pharmaceutical composition of rizatriptan contained in a unit dose upon administration to the female patients exhibits at least about 70% higher AUC₀₋₂ₕ, AUC₀₋ₜ or AUC_{0-refTmax} values compared to male patients.

In yet another aspect of the above embodiments, the present pharmaceutical composition of rizatriptan contained in a unit dose upon administration to the female patients exhibits at least about 60% higher AUC₀₋₂ₕ, AUC₀₋ₜ or AUC_{0-refTmax} values compared to male patients.

In yet another aspect of the above embodiments, the present pharmaceutical composition including rizatriptan contained in a unit dose upon administration to the female patients exhibits at least one of the following pharmacokinetic ratios compared to male patients:

a. Cₘₐₓ of about 1.9;
b. AUC_{0.2} of about 1.6;
c. AUC_{0-refTmax} of about 1.6; and

d. AUC₀₋ₜ of about 1.5.

In an embodiment, the present application relates to a method of administering a pharmaceutical composition including rizatriptan or pharmaceutically acceptable salts thereof contained in a unit dose to a patient in need thereof, wherein said unit dose comprises less than about 5 mg of rizatriptan base and said composition upon administration to female patients exhibits at least one of the following pharmacokinetic parameters:

a. Tₘₐₓ of about 7 minutes to about 9 minutes;

b. Cₘₐₓ of about 36 ng/ml to about 48 ng/ml;

c. AUC₀₋₂ₕ of about 28 ng·hr/ml to about 34 ng·hr/ml;

d. AUC₀₋ₜ of about 40 ng·hr/ml to about 53 ng·hr/ml; and

e. AUC_{0-refTmax} of about 17 ng·hr/ml to about 23 ng·hr/ml.

In an aspect of the above embodiments, the present method of treating a migraine headache in a patient comprises administering a pharmaceutical composition including rizatriptan contained in a unit dose, wherein said unit dose is a single use parenteral injector device.

In an aspect of the above embodiments, the present unit dose is parenterally administered using injecting device selected from auto-injector, syringe like injector, needle free injector or jet injector.

In another aspect of the above embodiments, the composition is parenterally administered.

In an aspect of the above embodiments, said unit dose is provided in a single use parenteral auto-injector device.

In another aspect of the above embodiments, the present unit dose is provided in a single use parenteral auto-injector device designed for subcutaneous or intramuscular injection.

In another aspect of the above embodiments, the present pharmaceutical composition further comprises at least one stabilizing agent.

In yet another aspect of the above embodiments, the present pharmaceutical composition further comprises at least one stabilizing agent comprising sodium chloride.

In an embodiment, the present application relates to a method of treating a migraine headache in a patient by administering a pharmaceutical composition including rizatriptan or pharmaceutically acceptable salts thereof contained in a unit dose, wherein said unit dose comprises less than about 5 mg of rizatriptan base, and said patient is non-responsive to commercially available oral triptans.

In another aspect of the above embodiments, the present method of treating a migraine headache in a patient by administering a pharmaceutical composition including rizatriptan contained in a unit dose, wherein said patient is non-responsive to commercially available oral rizatriptan dosage forms.

In another aspect of the above embodiments, the present method of treating a migraine headache in a patient by administering a pharmaceutical composition including rizatriptan contained in a unit dose, wherein said patient is non-responsive to commercially available injectable triptan dosage forms.

In another aspect of the above embodiments, the present method of treating a migraine headache in a patient by administering a pharmaceutical composition including rizatriptan contained in a unit dose, wherein said patient is non-responsive to commercially available injectable sumatriptan dosage forms.

In an aspect of the above embodiments, the patient is suffering from acute migraine with or without aura.

In an embodiment, the present application relates to a pharmaceutical composition comprising an aqueous solution of rizatriptan or pharmaceutically acceptable salts thereof and at least one stabilizing agent contained in a unit dose.

In another embodiment, the present application relates to a pharmaceutical composition comprising an aqueous solution of rizatriptan or pharmaceutically acceptable salts thereof and at least one stabilizing agent contained in a unit dose, wherein said rizatriptan and stabilizing agent are present in a millimolar ratio of not more than about 0.17:1.0.

In another embodiment, the present application relates to a pharmaceutical composition comprising an aqueous solution of rizatriptan or pharmaceutically acceptable salts thereof and at least one stabilizing agent contained in a unit dose, wherein said rizatriptan and stabilizing agent are present in a millimolar ratio of about 0.07:1.0 to about 0.17:1.0.

In an aspect of the above embodiments, the stabilizing agent comprises sodium chloride.

In an embodiment, the present application relates to a pharmaceutical composition comprising an aqueous solution of rizatriptan or pharmaceutically acceptable salts thereof and at least one stabilizing agent comprising sodium chloride contained in a unit dose.

In another embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0.

In another embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of about 0.07:1.0 to about 0.17:1.0.

In an aspect of the above embodiments, the present pharmaceutical composition including rizatriptan contained in a unit dose comprises less than about 5 mg of rizatriptan base.

In another aspect of the above embodiments, the present pharmaceutical composition including rizatriptan contained in a unit dose comprises less than about 4 mg of rizatriptan base.

In another aspect of the above embodiments, the present pharmaceutical composition including rizatriptan contained in a unit dose comprises about 3 mg of rizatriptan base.

In an embodiment, the present application relates to a pharmaceutical composition comprising an aqueous solution of rizatriptan or pharmaceutically acceptable salts thereof and at least one stabilizing agent comprising sodium chloride contained in a unit dose comprising less than about 5 mg of rizatriptan base, wherein said rizatriptan and sodium chloride are present in a millimolar ratio of not more than about 0.17:1.0.

In an aspect of the above embodiments, the rizatriptan or pharmaceutically acceptable salts thereof used in the present application include, but not limited to, pharmaceutically acceptable, pharmacologically active derivatives of rizatriptan, including both individual enantiomers of rizatriptan (dextrogyral and levogyral enantiomers) in their substantially pure form and their pharmaceutically acceptable salts, mixtures (in any ratio) of rizatriptan enantiomers and their pharmaceutically acceptable salts, and active metabolites of rizatriptan and their pharmaceutically acceptable salts. The solid state form of rizatriptan used in the composition is not critical. For example, rizatriptan can be amorphous or crystalline. Examples of pharmaceutically acceptable salts include, but not limited to, any of the salts or co-crystals of rizatriptan selected from benzoate, sulphate, citrate, phosphate, maleate, formate, acetate, hydrochloride, hydrobromide, nitrate, mesylate, succinate and the like. The salts may be in the form of solvate, hydrate, hemihydrates or anhydrous forms. The amount of pharmaceutically acceptable rizatriptan salt used in the present composition is equivalent or less than about 5 mg of rizatriptan base. For example, 3.6 mg of rizatriptan benzoate salt is equivalent to 2.5 mg of rizatriptan base.

In an aspect of the above embodiments, the present pharmaceutical composition comprising an aqueous solution of rizatriptan contained in a unit dose comprises rizatriptan benzoate equivalent to rizatriptan base present in an amount of about 0.5 mg/ml to about 6 mg/ml.

In an embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0, and said composition has an osmolality of from about 250 mOsmol/kg to about 350 mOsmol/kg.

In another embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0, and said composition has a pH value of about 4.0 to about 7.5.

In an aspect of above embodiments, the present pharmaceutical composition including rizatriptan is stable for at least about 6 months upon storage at 25° C. and 60% relative humidity (RH) or at 40° C. and 75% relative humidity (RH).

In another aspect of the above embodiments, the present pharmaceutical composition including rizatriptan has a total impurities of not more than about 1.5%. Impurities as used herein is understood in the art and refers to undesirable components.

In an aspect of above embodiments, the present pharmaceutical composition including rizatriptan is stable for at least about 6 months upon storage at 25° C. and 60% relative humidity (RH) or at 40° C. and 75% relative humidity (RH) and has a total impurities of not more than about 1.5%.

In an embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0, and said composition is stable for at least 6 months upon storage at 25° C./60% relative humidity (RH) or at 40° C. and 75% relative humidity (RH).

In another embodiment, the present application relates to a pharmaceutical composition comprising an aqueous solution of rizatriptan or pharmaceutically acceptable salts thereof and at least one stabilizing agent comprising sodium chloride contained in a unit dose, wherein said rizatriptan and sodium chloride are present in a millimolar ratio of not more than about 0.17:1.0, and said composition is stable for at least 6 months and has a total impurities of not more than about 1.5%.

In an aspect of the above embodiments, the present pharmaceutical composition including rizatriptan optionally further comprises at least one pharmaceutically acceptable excipient selected from, but are not limited to, tonicity modifiers, buffering agents, preservatives, antioxidants and the like or mixtures thereof.

The term “tonicity modifier” or “stabilizing agent” as used herein, refers to an agent that ensures the tonicity of the aqueous solution, i.e. maintaining the osmolality, which should be essentially the same as normal physiological fluids and thus prevent post-administration swelling or rapid absorption of the composition because of differential ion concentrations between the composition and physiological fluids.

Examples of the tonicity modifiers or stabilizing agents that can be used in the present application include, but are not limited to, sodium chloride, potassium chloride, calcium chloride, sodium lactate, ringer's solution, dextrose, sorbitol, mannitol, sucrose, maltose, trehalose, glycerine, amino acids, and the like or mixtures thereof. The amount of tonicity modifiers that can be used in the present application ranges from about 0.5% to about 5.0%, weight by volume of the composition.

In an aspect of the above embodiments, the present pharmaceutical composition including rizatriptan has an osmolality of about 250 mOsmol/kg to about 350 mOsmol/kg.

The term “buffering agent” as used herein, refers to an agent or a mixture of agents which can maintain pH and stability of a composition. The optimum system and pH range will depend on the nature of the drug (acid or base) and compatibility with other formulation ingredients.

Examples of the buffering agents that can be used for the present application include, but are not limited to, citric acid, sodium citrate, disodium hydrogen phosphate, potassium phosphate, acetic acid, sodium acetate dihydrate, sodium acetate trihydrate, di sodium edetate, sodium bicarbonate, sodium tartrate, sodium hydroxide and the like or mixtures thereof. The amount of buffering agents that can be used in the present application ranges from about 0% to about 3.5%, weight by volume of the composition.

In an aspect of the above embodiments, the present pharmaceutical composition including rizatriptan has a pH value of about 4.0 to about 7.5.

The term “preservative” as used herein, refers to an agent which provides a chemical means of preservation and extension of product shelf life by inhibiting microbial growth, and thereby, constraining decomposition of the drug or pharmaceutical composition.

Examples of the preservatives that can be used in the present application include, but are not limited to, benzalkonium chloride, benzyl alcohol, phenol, methyl paraben, ethyl paraben, propyl paraben, butyl paraben, and halogenate derivatives thereof, thimerosal, meta-cresol and the like or mixtures thereof. The amount of preservatives that can be used in the present application ranges from about 0% to about 1.0%, weight by volume of the composition.

The term “antioxidants” as used herein, refers to an agent that provides chemical or biological means of protection by prevention of oxidative degradation of the drug or pharmaceutical composition. Examples of the antioxidants that can be used in the present application include, but are not limited to, ascorbic acid, ethylene diamine tetra acetic acid (EDTA), sodium bisulfite, sodium metabisulfite, citric acid, tartaric acid, glycerol, alpha tocopherol and the like or mixtures thereof. The amount of antioxidants that may be used in the present application ranges from about 0% to about 1.0%, weight by volume of the composition.

In an aspect of the above embodiments, the present pharmaceutical composition including rizatriptan comprises pharmaceutically acceptable vehicle such as water for injection, ringer's solution, isotonic sodium chloride solution or mannitol.

The present pharmaceutical composition including rizatriptan optionally comprises “co-solvents”, referring to an agent used to enhance solubility of the drug. It also reduces dose volume and optimises insolubility. Examples of the co-solvents that can be used in the present application include, but are not limited to, propylene glycol, glycerol, low molecular weight poly ethylene glycols and the like or mixtures thereof. The amount of co-solvents that can be used in the present application ranges from about 0% to about 40%, weight by volume of the composition.

In an aspect of the above embodiments, the present pharmaceutical composition including rizatriptan is filterable through 0.2μ membrane filter.

The term “filterable” means a composition that has passed through a filter having a pore size sufficiently small to result the composition free or substantially free of bacterial contaminants. Bacteria generally range in size from about 0.2μ to about 600μ with most bacteria having a size in the range of about 1μ to about 10μ. Filters having pore size of about 0.2μ or less are considered to produce sterile filtrates and are sufficiently small to result in a filter sterilized composition.

In another aspect of the above embodiments, the present composition is prepared for administration to a patient suffering from acute migraine with or without aura.

In yet another aspect of the above embodiments, the present pharmaceutical composition including rizatriptan can also be co-administered (simultaneously or sequentially) with one or more pharmaceutical agents of value in treating a migraine headache or related disease conditions.

Examples of the pharmaceutical agents that can be co-administered are selected from, but not limited to, any analgesics like acetaminophen, any non-steroidal anti-inflammatory drugs (NSAIDs), such as aspirin, ibuprofen, tolfenamic acid or any COX-II inhibitors and the like or mixtures thereof.

In an embodiment, the present application relates to a process for preparing the present pharmaceutical composition including rizatriptan contained in a unit dose, which comprises mixing of rizatriptan or pharmaceutically acceptable salts thereof with suitable pharmaceutically acceptable excipients, adjusting volume using pharmaceutically acceptable vehicle, such as water for injection followed by filtration for sterilization, in-process check for pH and tonicity.

In another embodiment, the present application also relates to a process for preparing the present pharmaceutical composition including rizatriptan contained in a unit dose, which comprises mixing of rizatriptan or pharmaceutically acceptable salts thereof with suitable stabilizing agent comprising sodium chloride, adjusting volume using pharmaceutically acceptable vehicle, such as water for injection, followed by filtration for sterilization, in-process check for pH and tonicity.

In an aspect of the above embodiments, the process of preparing present pharmaceutical composition including rizatriptan comprises mixing of rizatriptan or pharmaceutically acceptable salts thereof with suitable stabilizing agent comprising sodium chloride in a millimolar ratio of about 0.07:1.0 to about 0.17:1.0.

In an aspect of the above embodiments, the resulting aqueous solution of rizatriptan for the present pharmaceutical composition as prepared by processes described herein above, can be filled in suitable container and closed by suitable closure. The present pharmaceutical composition including rizatriptan can be sterilized by various sterilization techniques available for parenteral dosage forms such as filtration, terminal sterilization using moist heat or irradiation and using aseptic techniques. The manufacturing process for the present application is similar to typical known manufacturing processes for sterile parenteral formulations.

In an aspect of the above embodiments, the parenteral composition of the present application comprises a sterile, pyrogen-free aqueous solution of rizatriptan or pharmaceutically acceptable salts thereof packaged in single-dose or multi-dose containers using suitable closures.

For the purpose of present pharmaceutical composition, containers are pharmaceutically acceptable, which are made of material which is non-reactive or substantially non-reactive with the parenteral formulation, selected from glass ampoules, bottles or vials, plastic bottles or bags, or prefilled syringes.

Further for the purpose of present pharmaceutical composition, closures are pharmaceutically acceptable, equipped with a firm seal to prevent entry of microorganisms and other contaminants and made up of components that should not react with the contents, selected from a sterile rubber, plastic or metal closures such as bromobutyl rubber, chlorobutyl rubber, a fluoropolymer, silicone, polyethylene, polypropylene, nylon, polyurethane, polyvinylchloride, polyacrylates, polycarbonates, and the like or mixtures thereof.

In an aspect of the above embodiments, the pharmaceutical composition of the present application is stable and remains clear, colorless or pale yellow and has total impurities of not more than about 1.5% for at least about 6 months when stored in pharmaceutically acceptable containers and closures and upon storage at 25° C. and 60% relative humidity (RH) or at 40° C. and 75% relative humidity (RH).

In an aspect of the above embodiments, the pharmaceutical composition of the present application comprises a sterile, pyrogen-free aqueous solution of rizatriptan or pharmaceutically acceptable salts thereof packaged in a unit dose comprising a single use parenteral injector device.

In an aspect of the above embodiments, the present unit dose is parenterally administered using injectable device selected from auto-injector, syringe like injector, needle free injector or jet injector.

In another aspect of the above embodiments, the unit dose is provided in a single use parenteral auto-injector device designed for subcutaneous or intramuscular injection.

In an embodiment, the present application relates to an auto-injector device comprising a unit dose of the pharmaceutical composition, as disclosed herein.

In an embodiment, the present application relates to an injector device comprising an aqueous solution of rizatriptan or its pharmaceutically acceptable salts and at least one stabilizing agent contained in a unit dose, wherein said rizatriptan and stabilizing agent are present in a millimolar ratio of not more than about 0.17:1.

In an aspect of the above embodiments, said unit dose comprises less than about 5 mg of rizatriptan base.

In an aspect of the above embodiments, said stabilizing agent comprises sodium chloride.

In an aspect of the above embodiments, said composition has a pH value of about 4 to about 7.5.

In an aspect of the above embodiments, said composition has an osmolality of from about 250 mOsmol/kg to about 350 mOsmol/kg.

In an aspect of the above embodiments, said composition is filterable through 0.2μ membrane filter.

In an aspect of the above embodiments, said composition is stable for at least about 6 months upon storage at 25° C. and at 60% relative humidity (RH) or 40° C. and 75% relative humidity (RH).

In an aspect of the above embodiments, said composition has total impurities of not more than about 1.5%.

In an aspect of the above embodiments, said composition is prepared for parenteral administration.

In an aspect of the above embodiments, the device is selected from auto-injector, syringe like injector, needle free injector, and jet injector.

In an aspect of the above embodiments, said unit dose is provided in a single use parenteral auto-injector device.

In an aspect of the above embodiments, said device is designed for subcutaneous or intramuscular injection.

In an aspect of the above embodiments, said composition is designed for administering to a patient suffering from acute migraine with or without aura.

In an aspect of the above embodiments, said composition is designed for administering to a patient suffering from acute migraine with aura.

In an aspect of the above embodiments, said composition is designed for administering to a patient suffering from acute migraine without aura.

In an aspect of the above embodiments, said composition exhibits Tmax value of at least about 80% less compared to commercially available oral rizatriptan compositions.

In an aspect of the above embodiments, said Tₘₐₓ value is about 8 minutes to about 12 minutes.

In an aspect of the above embodiments, said composition provides at least about 70% of headache relief in about 30 minutes.

In an aspect of the above embodiments, said composition provides at least about 80% of headache relief in about 60 minutes.

In an aspect of the above embodiments, said device of claim 59, wherein said composition upon administration exhibits at least one of the following pharmacokinetic parameters:
(a) Tₘₐₓ value of not more than about 12 minutes;
(b) Cₘₐₓ of not more than about 37 ng/ml;
(c) AUC₀₋₂ₕ of not more than about 31 ng·hr/ml;
(d) AUC₀₋ₜ of not more than about 44 ng·hr/ml; and
(e) AUC_{0-refTmax} of not more than about 19 ng·hr/ml.

In an aspect of the above embodiments, said composition exhibits AUC_{0-refTmax} of about 60% higher than commercially available oral rizatriptan compositions.

In an aspect of the above embodiments, said composition exhibits at least about 60% less Tmax value for female patients compared to male patients.

In an aspect of the above embodiments, said composition exhibits at least about 60% high Cmax value for female patients compared to male patients.

In an embodiment, the present application relates to an injector device comprising a unit dose of a pharmaceutical composition including rizatriptan or a pharmaceutically acceptable salt thereof, wherein said composition upon administration exhibits Tmax of not more than about 12 minutes, and said unit dose comprises at least about 60% less rizatriptan compared to commercially available oral rizatriptan compositions.

In an aspect of the above embodiments, said unit dose comprises less than about 5 mg of rizatriptan base.

In an aspect of the above embodiments, said composition exhibits Tmax value of at least about 80% less compared to commercially available oral rizatriptan compositions.

In an aspect of the above embodiments, said Tmax value is about 8 minutes to about 12 minutes.

In an aspect of the above embodiments, said composition provides at least about 70% of headache relief in about 30 minutes.

In an aspect of the above embodiments, said composition provides at least about 80% of headache relief in about 60 minutes.

In an aspect of the above embodiments, said composition is parenterally administered.

In an aspect of the above embodiments, the device is selected from auto-injector, syringe like injector, needle free injector, and jet injector.

In an aspect of the above embodiments, said unit dose is provided in a single use parenteral auto-injector device.

In an aspect of the above embodiments, said device is designed for subcutaneous or intramuscular injection.

In an aspect of the above embodiments, said composition is designed for administering to a patient suffering from acute migraine with or without aura.

In an aspect of the above embodiments, said composition is designed for administering to a patient suffering from acute migraine with aura.

In an aspect of the above embodiments, said composition is designed for administering to a patient suffering from acute migraine without aura.

In an aspect of the above embodiments, said composition further comprises at least one stabilizing agent.

In an aspect of the above embodiments, said stabilizing agent comprises sodium chloride.

In an aspect of the above embodiments, said composition has a pH value of about 4 to about 7.5.

In an aspect of the above embodiments, said composition has an osmolality of from about 250 mOsmol/kg to about 350 mOsmol/kg.

In an aspect of the above embodiments, said composition is filterable through 0.2μ membrane filter.

In an aspect of the above embodiments, said composition is stable for at least about 6 months upon storage at 25° C. and at 60% relative humidity (RH) or 40° C. and 75% relative humidity (RH).

In an aspect of the above embodiments, said composition has total impurities of not more than about 1.5%.

In an embodiment, the present application relates to an injector device comprising a unit dose of a pharmaceutical composition including rizatriptan or a pharmaceutically acceptable salt thereof, wherein said unit dose comprises less than about 5 mg of rizatriptan base and said composition upon administration exhibits at least one of the following pharmacokinetic parameters:

(a) Tmax value of not more than about 12 minutes;

(b) Cmax of not more than about 37 ng/ml;

(c) AUC0-2 h of not more than about 31 ng·hr/ml;

(d) AUC0-t of not more than about 44 ng·hr/ml; and

(e) AUC0-refTmax of not more than about 19 ng·hr/ml.

In an aspect of the above embodiments, said composition exhibits AUC0-refTmax of about 60% higher than commercially available oral rizatriptan compositions.

In an aspect of the above embodiments, said composition exhibits at least about 60% less Tmax value for female patients compared to male patients.

In an aspect of the above embodiments, said composition exhibits at least about 60% high Cmax value for female patients compared to male patients.

In an aspect of the above embodiments, said composition is designed for administering to a patient suffering from acute migraine with or without aura.

In an aspect of the above embodiments, said composition is designed for administering to a patient suffering from acute migraine with aura.

In an aspect of the above embodiments, said composition is designed for administering to a patient suffering from acute migraine without aura.

In an aspect of the above embodiments, said composition is parenterally administered.

In an aspect of the above embodiments, the device is selected from auto-injector, syringe like injector, needle free injector, and jet injector.

In an aspect of the above embodiments, said unit dose is provided in a single use parenteral auto-injector device.

In an aspect of the above embodiments, said device is designed for subcutaneous or intramuscular injection.

In an aspect of the above embodiments, said composition further comprises at least one stabilizing agent.

In an aspect of the above embodiments, said stabilizing agent comprises sodium chloride.

In an aspect of the above embodiments, said composition has a pH value of about 4 to about 7.5.

In an aspect of the above embodiments, said composition has an osmolality of from about 250 mOsmol/kg to about 350 mOsmol/kg.

In an aspect of the above embodiments, said composition is filterable through 0.2μ membrane filter.

In an aspect of the above embodiments, said composition is stable for at least about 6 months upon storage at 25° C. and at 60% relative humidity (RH) or 40° C. and 75% relative humidity (RH).

In an aspect of the above embodiments, said composition has total impurities of not more than about 1.5%.

The present application is further illustrated by the examples which are provided merely to be exemplary of the pharmaceutical composition described above and do not limit the scope of the application. Certain modifications and equivalents will be apparent to those skilled in the art and are intended to be included within the scope of the present application.

The present invention is illustrated below by reference to the following examples. However, one skilled in the art will appreciate that the specific methods and results discussed are merely illustrative of the present invention, and not to be construed as limiting the application. The following examples may include compilations of data that are representative of data gathered at various times during the course of development and experimentation related to the present invention.

### EXAMPLES

### Examples 1-9

The pharmaceutical composition comprising rizatriptan or pharmaceutically acceptable salts thereof were prepared as given in Table 1.

TABLE 1

| Composition | Ex 1 | Ex 2 | Ex 3 | Ex 4 | Ex 5 | Ex 6 | Ex 7 | Ex 8 | Ex 9 |
|---|---|---|---|---|---|---|---|---|---|
| Rizatriptan | 2.5 mg/ | 2.5 mg/ | 2.5 mg/ | 2.5 mg/ | 2.5 mg/ | 2.5 mg/ | 2.5 mg/ | l.5 mg/ | 3.0 mg/ |
| benzoate equivalent | 0.5 ml | 0.5 ml | 0.5 ml | 0.5 ml | 0.5 ml | 0.5 ml | 0.5 ml | 0.5 ml | 0.5 ml |
| to Rizatriptan Base | 0.73 | 0.73 | 0.73 | 0.73 | 0.73 | 0.73 | 0.73 | 0.43 | 0.87 |
| Sodium Chloride | 0.8 | — | — | — | — | 0.8 | 0.55 | 0.85 | 0.8 |
| (% w/v) | | | | | | | | | |
| Dextrose | — | 4.5 | 4.3 | 3.1 | — | — | — | — | — |
| anhydrous (% w/v) | | | | | | | | | |
| Disodium EDTA | 0.2 | — | 0.2 | — | — | — | — | — | — |
| (% w/v) | | | | | | | | | |
| Sodium Acetate | — | — | — | 0.598 | — | — | 0.598 | — | — |
| Trihydrate (% w/v) | | | | | | | | | |
| Acetic Acid (2N) | — | — | — | 0.3 | — | — | 0.3 | — | — |
| (% w/v) | | | | | | | | | |
| Potassium | — | — | — | — | 0.1128 | — | — | — | — |
| Phosphate | | | | | | | | | |
| Monobasic (% w/v) | | | | | | | | | |
| Sodium Hydroxide | — | — | — | — | 0.0749 | — | — | — | — |
| (% w/v) | | | | | | | | | |
| Water For Injection | q.s. | q.s. | q.s. | q.s. | q.s. | q.s. | q.s. | q.s. | q.s. |
| pH | 5.3 | 6.72 | 5.45 | 6.14 | 7.05 | 6.68 | 5.46 | 6.71 | 6.53 |
| Osmolality | 297 | 293 | 296 | 282 | 289 | 287 | 296 | — | 296 |
| (mOsmol/kg) | | | | | | | | | |

Procedure:
1. Accurately weighed quantities of the drug and excipients were dissolved in water for injection under continuous stirring to get clear solution.
2. The step 1 solution was then filtered aseptically through the 0.22μ filter membrane.
3. Required volume of 0.5 ml and required pH were adjusted for the step 2 solution.
4. The step 3 solution was then filled in glass vial and closed with rubber stopper.

### Example 10

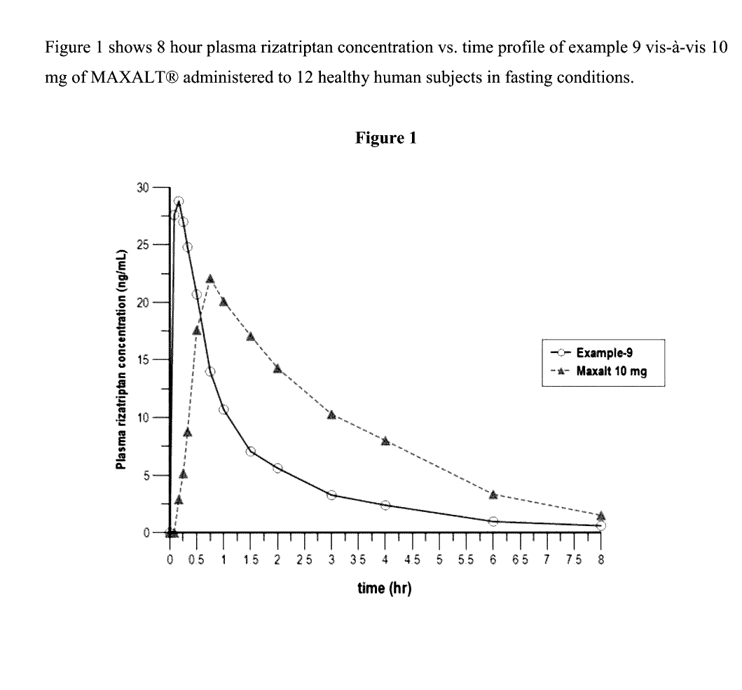

The pharmacokinetic parameters for pharmaceutical compositions of the present application were studied in comparison with MAXALT® (10 mg) oral tablets by using a two-way crossover method. The study was conducted in total 12 healthy human subjects—6 females and 6 males, in fasting condition and the subjects were subcutaneously administered a single dose of composition of Example 9 equivalent to 3 mg of rizatriptan. The results are shown in below Table 2 and the mean plasma rizatriptan concentration vs. time profile vis-a-vis MAXALT® is shown in FIG. 1.

TABLE 2

| Param- | | Example 9 | Example 9 | MAXALT ® |
|---|---|---|---|---|
| eters | Example 9 | (females) | (males) | (10 mg) |
| C_{max (ng/ml)} | 32.0 ± | 41.9 ± 10.7 | 22.2 ± 5.92 | 24.9 ± 7.11 |
| | 13.2 | | | |
| Tₘₐₓ (hr) | 0.17 | 0.13 | 0.30 | 0.75 |
| AUC_{(0-2 h)} | 26.7 ± | 32.7 ± 5.05 | 20.6 ± 4.94 | 30.4 ± 8.53 |
| (ng · hr/ml) | 7.90 | | | |
| AUC₍₀₋ₜ₎ | 37.8 ± | 45.9 ± 7.39 | 29.7 ± 7.14 | 67.9 ± 23.9 |
| (ng · hr/ml) | 10.9 | | | |
| AUC_{(0-∞)} | 39.6 ± | 47.6 ± 7.13 | 31.6 ± 6.91 | 70.9 ± 24.2 |
| (ng · hr/ml) | 10.7 | | | |
| AUC_{(0-refTmax)} | 16.1 ± | 20.2 ± 3.44 | 11.9 ± 3.60 | 8.10 ± 3.71 |
| (ng · hr/ml) | 5.48 | | | |

*Tmax is given in median value and all other parameters are given in mean value.

### Example 11

The response of % headache relief for MAXALT® 10 mg was simulated from an exposure-response model (Tokuoka et al.) and the simulated values were validated against % headache relief values of US FDA approved label for MAXALT® 10 mg. The validated exposure-response model was used for simulating the response of % headache relief for the composition of Example 9. All the results are shown in below table 3.

TABLE 3

| | % headache | | |
|---|---|---|---|
| | relief for | | |
| | MAXALT ® (as | | % headache |
| Time | per approved | % headache | relief predicted |
| (Min) | label) | relief predicted | for Ex-9 |
| 30 | 20 | 22 | 71 |
| 60 | 45 | 48 | 80 |
| 90 | 66 | 60 | 83 |
| 120 | 72 | 67 | 84 |
| 180 | 76 | 74 | 86 |
| 240 | 78 | 78 | 86 |

### Example 12

The pharmaceutical composition comprising rizatriptan or pharmaceutically acceptable salts thereof and sodium chloride present in millimolar ratio of about 0.01:1.0 to about 0.17:1.0 were prepared as steps shown above and presented in below Table 4. The prepared pharmaceutical compositions were studied for physical and chemical stability at 25° C./65 RH and at 40° C./75 RH for 6 months and the results are shown in below Tables 5, 6 and 7.

TABLE 4

| | Millimolar ratios | | | | | |
|---|---|---|---|---|---|---|
| | 0.01:1.0 | 0.02:1.0 | 0.04:1.0 | 0.07:1.0 | 0.13:1.0 | 0.16:1.0 |
| Composition | Ex 12A | Ex 12B | Ex 12C | Ex 12D | Ex 12E | Ex 12F |
| Rizatriptan | 0.07 | 0.14 | 0.29 | 0.43 | 0.73 | 0.87 |
| benzoate | | | | | | |
| (% w/v) | | | | | | |
| Sodium | 0.90 | 0.90 | 0.90 | 0.85 | 0.80 | 0.80 |
| Chloride | | | | | | |
| (% w/v) | | | | | | |

| | |
|---|---|
| Water For | q.s. to 0.5 ml |
| Injection | |

TABLE 5

| | Ex 12A | | | | | Ex 12B | | | | |
|---|---|---|---|---|---|---|---|---|---|---|
| | | 25° C./60% RH | | 40° C./75% RH | | | 25° C./60% RH | | 40° C./75% RH | |
| Parameters | Initial | 3 M* | 6 M | 3 M | 6 M | Initial | 3 M | 6 M | 3 M | 6 M |
| Description | Clear | Pass | Pass | Pass | Pass | Clear | Pass | Pass | Pass | Pass |
| | colorless | | | | | colorless | | | | |
| | liquid | | | | | liquid | | | | |
| Assay (% w/v) | 102.0 | 101.1 | 101.1 | 100.6 | 100.1 | 102.1 | 101.1 | 99.6 | 99.7 | 97.7 |
| Single Highest | 0.04 | 0.08 | 0.10 | 0.22 | 0.57 | 0.02 | 0.05 | 0.10 | 0.26 | 0.54 |
| Unknown Impurity | | | | | | | | | | |
| Total Impurities | 0.17 | 0.33 | 0.37 | 0.61 | 1.22 | 0.11 | 0.26 | 0.36 | 0.72 | 1.27 |
| pH | 6.60 | 7.92 | 8.00 | 8.13 | 8.12 | 6.73 | 7.75 | 7.50 | 7.86 | 7.89 |
| Osmolality | 291 | — | — | — | — | 294 | — | — | — | — |
| (mOsmol/Kg) | | | | | | | | | | |

*M = Month

TABLE 6

| | Ex 12C | | | | | Ex 12D | | | | |
|---|---|---|---|---|---|---|---|---|---|---|
| | | 25° C./60% RH | | 40° C./75% RH | | | 25° C./60% RH | | 40° C./75% RH | |
| Parameters | Initial | 3 M | 6 M | 3 M | 6 M | Initial | 3 M | 6 M | 3 M | 6 M |
| Description | Clear | Pass | Pass | Pass | Pass | Clear | Pass | Pass | Pass | Pass |
| | colorless | | | | | colorless | | | | |
| | liquid | | | | | liquid | | | | |
| Assay (% w/v) | 101.7 | 101.2 | 102.6 | 99.1 | 100.1 | 98.9 | 98.9 | 99.8 | 97.3 | 97.0 |
| Single Highest | 0.02 | 0.05 | 0.07 | 0.20 | 0.48 | 0.02 | 0.04 | 0.08 | 0.21 | 0.41 |
| Unknown Impurity | | | | | | | | | | |
| Total Impurities | 0.09 | 0.25 | 0.35 | 0.84 | 1.42 | 0.09 | 0.24 | 0.36 | 0.80 | 1.57 |
| pH | 6.70 | 7.32 | 7.30 | 7.49 | 7.54 | 6.71 | 7.22 | 7.30 | 7.23 | 7.00 |
| Osmolality | 302 | — | — | — | — | 290 | — | — | — | — |
| (mOsmol/Kg) | | | | | | | | | | |

TABLE 7

| | Ex 12E | | | | | Ex 12F | | | | |
|---|---|---|---|---|---|---|---|---|---|---|
| | | 25° C./60% RH | | 40° C./75% RH | | | 25° C./60% RH | | 40° C./75% RH | |
| Parameters | Initial | 3 M* | 6 M | 3 M | 6 M | Initial | 3 M | 6 M | 3 M | 6 M |
| Description | Clear | Pass | Pass | Pass | Pass | Clear | Pass | Pass | Pass | Pass |
| | colorless | | | | | colorless | | | | |
| | liquid | | | | | liquid | | | | |
| Assay (% w/v) | 99.9 | 101.2 | 100.6 | 99.5 | 98.8 | 99.9 | 100.7 | 99.78 | 98.4 | 93.6 |
| Single Highest | 0.03 | 0.03 | 0.04 | 0.08 | 0.12 | 0.03 | 0.06 | 0.04 | 0.27 | — |
| Unknown Impurity | | | | | | | | | | |
| Total Impurities | 0.06 | 0.13 | 0.24 | 0.31 | 0.49 | 0.06 | 0.24 | 0.22 | 1.35 | — |
| pH | 6.53 | 7.15 | 6.68 | 6.62 | 7.2 | 6.53 | 7.02 | 6.8 | 6.73 | 6.25 |
| Osmolality | 296 | 276 | 309 | 302 | 291 | 296 | 290 | 306 | 292 | 311 |
| (mOsmol/Kg) | | | | | | | | | | |

While several particular forms of the application have been illustrated and described, it will be apparent that various modifications and combinations of the application detailed in the text can be made without departing from the spirit and scope of the application.

We claim:

1. A method of treating migraine headache in a patient in need thereof comprising administering to the patient a pharmaceutical composition including rizatriptan or a pharmaceutically acceptable salt thereof contained in a unit dose, wherein said composition upon administration exhibits Tₘₐₓ of not more than about 12 minutes, and said unit dose comprises at least about 60% less rizatriptan as compared to commercially available oral rizatriptan compositions.

2. The method of claim 1, wherein said unit dose comprises less than about 5 mg of rizatriptan base.

3. The method of claim 1, wherein said composition exhibits Tₘₐₓ value of at least about 60% less as compared to commercially available oral rizatriptan compositions.

4. The method of claim 3, wherein said Tₘₐₓ value is about 8 minutes to about 12 minutes.

5. The method of claim 1, wherein said composition provides at least about 70% of headache relief in about 30 minutes.

6. The method of claim 1, wherein said composition provides at least about 80% of headache relief in about 60 minutes.

7. The method of claim 1, wherein said composition is parenterally administered.

8. The method of claim 1, wherein said unit dose is provided in a single use parenteral auto-injector device.

9. The method of claim 8, wherein said device is designed for subcutaneous or intramuscular injection.

10. The method of claim 1, wherein said patient is suffering from acute migraine with or without aura.

11. The method of claim 1, wherein said composition further comprises at least one stabilizing agent.

12. A method of treating migraine headache in a patient in need thereof comprising administering to the patient a pharmaceutical composition including rizatriptan or a pharmaceutically acceptable salt thereof contained in a unit dose, wherein said unit dose comprises less than about 5 mg of rizatriptan base and said composition upon administration exhibits at least one of the following pharmacokinetic parameters:
(a) Tₘₐₓ value of not more than about 12 minutes;
(b) Cₘₐₓ of not more than about 37 ng/ml;
(c) AUC₀₋₂ₕ of not more than about 31 ng·hr/ml;
(d) AUC₀₋ₜ of not more than about 44 ng·hr/ml; and
(e) AUC_{0-refTmax} of not more than about 19 ng·hr/ml.

13. The method of claim 12, wherein said composition exhibits AUC_{0-refTmax} of at least about 60% higher than commercially available oral rizatriptan compositions.

14. The method of claim 12, wherein said composition exhibits at least about a 60% lower Tₘₐₓ value for female patients compared to male patients.

15. The method of claim 12, wherein said composition exhibits at least about a 60% higher Cₘₐₓ value for female patients compared to male patients.

16. The method of claim 12, wherein said patient is suffering from acute migraine with or without aura.

17. The method of claim 12, wherein said composition is parenterally administered.

18. The method of claim 12, wherein said unit dose is provided in a single use parenteral auto-injector device.

19. The method of claim 17, wherein said device is designed for subcutaneous or intramuscular injection.

20. The method of claim 12, wherein said composition further comprises at least one stabilizing agent.

21. A pharmaceutical composition comprising an aqueous solution of rizatriptan or pharmaceutically acceptable salts thereof and at least one stabilizing agent contained in a unit dose, wherein said rizatriptan and stabilizing agent are present in a millimolar ratio of not more than about 0.17:1.0.

22. The composition of claim 21, wherein said unit dose comprises less than about 5 mg of rizatriptan base.

23. The composition of claim 21, wherein said stabilizing agent comprises sodium chloride.

24. The composition of claim 21, wherein said composition has a pH value of about 4 to about 7.5.

25. The composition of claim 21, wherein said composition has an osmolality of from about 250 mOsmol/kg to about 350 mOsmol/kg.

26. The composition of claim 21, wherein said composition is filterable through 0.2μ membrane filter.

27. The composition of claim 21, wherein said composition is stable for at least about 6 months upon storage at 25° C. and at 60% relative humidity (RH) or 40° C. and 75% relative humidity (RH).

28. The composition of claim 21, wherein said composition has total impurities of not more than about 1.5%.

29. The composition of claim 21, wherein said composition is prepared for parenteral administration.

30. The composition of claim 21, wherein said composition is designed for subcutaneous or intramuscular injection.
